(19) United States
(12) Plant Patent Application Publication
(10) Pub. No.: US 2005/0246811 P1
(43) Pub. Date: Nov. 3, 2005

(54) Carnation plant called "CFPC Ruby"

(50) DIANTHUS CARYOPHYLLUS; CARNATION

(76) Inventor: Oscar T. Hasegawa, Salinas, CA (US)

Correspondence Address: CHRISTIE, PARKER & HALE, LLP, PO BOX 7068, PASADENA, CA, 91109-7068, US

(21) Appl. No.: 10/837,430

(22) Filed: Apr. 30, 2004

(51) Int. Cl. A01H 5/00

(52) U.S. Cl. PLT/278

(57) ABSTRACT

A new and distinct variety of carnation plant characterized by the raspberry color of the flower.

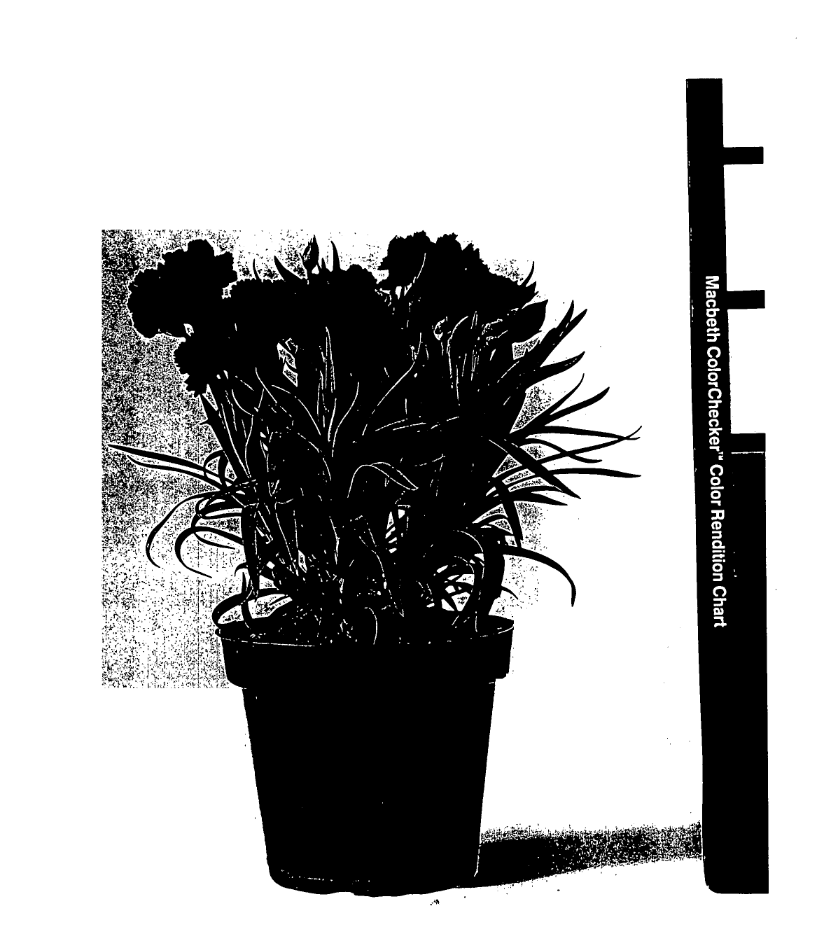

### BOTANICAL CLASSIFICATION

The present invention relates to a new and distinct variety of hybrid Dianthus caryophyllys. The varietal denominator of the new variety is ‘CFPC Ruby’.

### BACKGROUND OF THE INVENTION

The present invention relates to a new and distinct variety of carnation plant, named ‘CFPC Ruby’, which is a sport of the cultivar CFPC Selena (patented; No. PP11,899), a crimson colored commercially available carnation of the Adorable CFPC Cotillion family. The primary objective of the controlled breeding program was to produce a cultivar having a flower of new color and to expand the color range in the CFPC Cotillion family.

### SUMMARY OF THE INVENTION

Among the novel characteristics possessed by the new variety which distinguishes it is a distinctive raspberry flower color which retains its color without fading. When grown in 4½ inch pots in Salinas, Calif., plant diameter averages 6½ inch. Pinched plants are about 6¾ inches tall and flowers have about 1¾-2 inches average diameter; the crown is higher than ¾ inch. Foliage is about 3⅜ inches long and about 5/16 inches wide. The bloom has a tendency to split calyx at maturity.

Asexual reproduction by propagation by side shoot cuttings of the new variety as performed in Salinas, Calif., shows that the foregoing and other distinguishing characteristics come true to form and are established and transmitted through succeeding propagations.

### BRIEF DESCRIPTION OF ILLUSTRATION

The accompanying photographic illustration shows a typical specimen of the new variety in color as nearly true as it is reasonably possible to make in a color illustration of this character.

### DESCRIPTION OF THE NEW VARIETY

Referring now specifically to the new and distinct variety of carnation plant the following is a detailed description of ‘CFPC Ruby’ with color description where indicated by reference to The Royal Horticultural Society Colour Chart, except where common terms of color definition are employed. Color designation and other values stated may deviate slightly from the stated values from flowering to flowering, but the deviations will be within the range expected from varying environmental, seasonal and cultural conditions.

The new variety of carnation plant as herein described may vary in slight detail due to climatic, soil and cultural conditions under which the variety may be grown, the present description being of the variety as grown inside flower testing facilities at Salinas, California. The height of the plant and flower and foliage size were all established as an average for a 115-day old plant using standard commercial cultural practices. The data accurately characterizes the new cultivar in question.

The new variety is a greenhouse-type plant, suitable for production as a pot spray.
Plant:
   Height.—About 6¾ inches tall.
   Growing habit.—Semi-upright.
   Blooming habit.—As a spray.
   Rooting time.—About 17 days.
   Breaking action.—Very good.
   Mutation prone.—No.
   Disease Resistance.—Good.
Foliage:
   Color.—Upper side: near 138A; lower side: near 137D.
   Size.—Length: about 3⅜ inches; width: about 5/16 inches.
   Stem color.—Near 137D.
   Stem diameter.—Base: about ¼ inches; Apex: about 1/16 inches.
   Leaf base.—Color: near 138D; Shape: straight, smooth edge.
   Leaf apex.—Color: near 138A; Shape: smooth edge, lanceolate.
Bud:
   Shape.—Ovoid.
   Color.—Near 138B.
   Size.—length: about 1 inch; diameter: about ⅜ inches.
Calyx:
   No. of lobes (sepals).—Five.
   Size.—Length: about ¾ inches; width: about 5/16 inches.
   Color.—Outside: near 138B; inside: near 139D.
Bloom:
   Size.—Diameter (average): about 1¾ inches; depth (average): about ¾ inches.
   Borne.—Terminally.
   Color.—General tonality: raspberry (purplish red); Upper side: close to 57A; Lower side: close to 57B; Center: close to 57A Outer petals: close to 57A.
   Keeping quality.—Good.
   Fragrance.—None to slight.
Petals:
   Number.—Thirty six.
   Outer petals.—Length: about 1 11/16 inches;
   Width: about ¾ inches.
   Central petals.—Length: about 1¼ inches; Width: about ⅜ inches.
   Texture.—Smooth.
   Appearance.—Shiny.
   Form.—Fan-shaped.
   Edge.—Fine serration.
Reproductive organs:
   Stamen.—None (male sterile).
   Anthers.—None.
   Filaments.—None.
   Pistil.—Length: About 1⅛ inches; Number: one (four lobed) Base Color: White (whiter than 155D); Top: ⅛-¼ inch, color: tinged 57B.
   Stigma.—Number: 4; Base Color: White (whiter than 155D) Top: ⅛-¼ inch, color: tinged 57B.
   Fertility.—Male: sterile; Female: Good.
   Ovary.—Shape: conical; Color: base 155D, body 145A, apex 145A.
   Size.—Length: about ⅝ inches; Width: about ¼ inches.
Fruit and seed: None produced.

1. A new and distinct variety of carnation plant identified as ‘CFPC Ruby’, substantially as shown and described.
